(19) United States
(12) Patent Application Publication
(10) Pub. No.: US 2022/0033477 A1
(43) Pub. Date: Feb. 3, 2022

(54) LIQUID COMPOSITION FOR ORAL INGESTION CONTAINING COLLAGEN PEPTIDE AND METHOD FOR SUPPRESSING FOAMING OF LIQUID COMPOSITION FOR ORAL INGESTION

(71) Applicant: SUNTORY HOLDINGS LIMITED, Osaka-shi, Osaka (JP)

(72) Inventors: Wakana Taguchi, Soraku-gun, Kyoto (JP); Takako Imao, Soraku-gun, Kyoto (JP); Naoki Kasajima, Soraku-gun, Kyoto (JP); Megumi Endo, Soraku-gun, Kyoto (JP)

(73) Assignee: SUNTORY HOLDINGS LIMITED, Osaka-shi, Osaka (JP)

(21) Appl. No.: 17/297,885

(22) PCT Filed: Nov. 29, 2019
(86) PCT No.: PCT/JP2019/046712

§ 371 Date: May 27, 2021

(30) Foreign Application Priority Data
Nov. 30, 2018 (JP) 2018-226087

(51) Int. Cl. C07K 14/78; A61K 9/00; A23L 2/66; A23L 2/62; A23L 33/18

(52) U.S. Cl. CPC C07K 14/78; A61K 9/0095; A23V 2002/00; A23L 2/62; A23L 33/18; A23L 2/66

(57) ABSTRACT

The present invention aims to provide a technique that can suppress foaming of liquid oral compositions containing collagen peptides. The present invention relates to a liquid oral composition containing collagen peptides containing Pro-Hyp, wherein an amount of Pro-Hyp is 1.0 to 180 mg/100 mL, and the like.

### TECHNICAL FIELD

The present invention relates to a liquid oral composition containing collagen peptides. The present invention also relates to a method of suppressing foaming of a liquid oral composition containing collagen peptides. The present invention also relates to a method of producing a liquid oral composition and the like.

### BACKGROUND ART

Collagen is a protein that has been widely used as gelatin in the food field. Generally, it is believed hard to use a high molecular weight collagen efficiently in the body when it is orally ingested. Recently, collagen peptides, which are low molecular weight collagen molecules broken down from high molecular weight collagen molecules, have been developed to be suitable for ingestion in the body. Beverages containing collagen peptides have also been developed.

Proteins are generally known to easily make foam. For example, Patent Literature 1 discloses a foaming agent containing soy whey protein.

### CITATION LIST

### Patent Literature

Patent Literature 1: JP 2015-528703 T

### SUMMARY OF INVENTION

### Technical Problem

Liquid oral compositions containing collagen peptides easily make foam when shaken or the like. Thus, when a liquid oral composition containing collagen peptides is provided as a packaged beverage (a beverage in a container), for example, foaming occurs during filling of the container. When foaming occurs during filling of the container with the liquid oral composition, the foam squirts before the container is filled with a predetermined amount of the composition, interfering with the filling. When a packaged beverage containing collagen peptides produces a considerable amount of foam when shaken during transportation or the like, the foam may squirt when the container is opened, causing discomfort to consumers in some cases. In addition, when the beverage or the like is in the form of foam, consumers may be concerned about the quality even when the quality is unaffected. Thus, a technique that can suppress foaming of liquid oral compositions containing collagen peptide is useful, for example, in the production of beverages containing collagen peptides.

The present invention aims to provide a technique that can suppress foaming of liquid oral compositions containing collagen peptides.

### Solution to Problem

As a result of repeated studies on techniques that can suppress foaming of liquid oral compositions containing collagen peptides, the present inventors found that foaming of a liquid oral composition containing collagen peptides can be suppressed when the amount of Pro-Hyp (prolylhydroxyproline, hereinafter also referred to as PO) in the liquid oral composition is set to a specific amount. No reports have been made on the effect of Pro-Hyp, which is a dipeptide containing hydroxyproline (Hyp), on suppressing foaming.

Specifically, the present invention relates to the following liquid oral compositions and the following method of suppressing foaming of a liquid oral composition.

(1) A liquid oral composition containing: collagen peptides containing Pro-Hyp, wherein an amount of Pro-Hyp is 1.0 to 180 mg/100 mL.

(2) The liquid oral composition according to (1) above, wherein the amount of Pro-Hyp is 10 to 180 mg/100 mL.

(3) The liquid oral composition according to (1) or (2) above, wherein the liquid oral composition is a packaged beverage.

(4) A method of suppressing foaming of a liquid oral composition containing collagen peptides, the method including: adjusting an amount of Pro-Hyp in the liquid oral composition to 1.0 to 180 mg/100 mL.

(5) A method of producing a liquid oral composition, the method including: mixing collagen peptides with an aqueous medium so that an amount of Pro-Hyp is 1.0 to 180 mg based on 100 mL of the liquid oral composition.

### Advantageous Effects of Invention

The present invention can suppress foaming of a liquid oral composition containing collagen peptides.

### DESCRIPTION OF EMBODIMENTS

### <Liquid Oral Composition>

The liquid oral composition of the present invention contains collagen peptides containing Pro-Hyp, wherein an amount of Pro-Hyp is 1.0 to 180 mg/100 mL.

In the present invention, Pro-Hyp (PO) refers to a peptide (dipeptide) consisting of an amino acid sequence represented by proline-hydroxyproline. Herein, a peptide is described according to a conventional method such that the N-terminus (amino terminus) is on the left side and the C-terminus (carboxy terminus) is on the right side. In the present invention, preferably, Pro-Hyp is a linear dipeptide.

The liquid oral composition of the present invent ion contains collagen peptides. In the liquid oral composition of the present invention, the amount of Pro-Hyp is 1.0 to 180 mg/100 mL.

When the amount (concentration) of Pro-Hyp in the liquid oral composition containing collagen peptides is 1.0 to 180 mg/100 mL, foaming (bubbling) of the liquid oral composition is suppressed. In order to suppress foaming, the amount of Pro-Hyp in the liquid oral composition is preferably 5.0 mg/100 mL or more, more preferably 10 mg/100 mL or more. The amount of Pro-Hyp in the liquid oral composition is also preferably 40 mg/100 mL or more. In one embodiment, the amount of Pro-Hyp in the liquid oral composition is preferably 150 mg/100 mL or less, more preferably 100 mg/100 mL or less, still more preferably 80 mg/100 mL or less, particularly preferably 70 mg/100 mL or less. Herein, the range may be any combination of any upper limit and any lower limit. In one embodiment, the amount of PO in the liquid oral composition is preferably 5.0 to 180 mg/100 mL, more preferably 10 to 180 mg/100 mL, still more preferably 10 to 150 mg/100 mL, yet still more preferably 10 to 100 mg/100 mL, particularly preferably 10 to 80 mg/100 mL, most preferably 10 to 70 mg/100 mL. In another embodiment, the amount of PO in the liquid oral composition is preferably 5.0 to 150 mg/100 mL, more preferably 40 to 150 mg/100 mL.

For example, the liquid oral composition of the present invention may be prepared such that the amount of Pro-Hyp in the liquid oral composition is adjusted to the above ranges by using collagen peptides containing Pro-Hyp or by adding Pro-Hyp to collagen peptides. Collagen peptides containing Pro-Hyp may consist of Pro-Hyp, but usually contains other peptides in addition to Pro-Hyp. In the present invention, the source of Pro-Hyp is not limited. Pro-Hyp can be prepared by a method known in the relevant field, and may be prepared by suitably purifying collagen peptides containing Pro-Hyp. Pro-Hyp can also be produced by chemical synthesis. In the present invention, Pro-Hyp is included in collagen peptides, regardless of its source.

Collagen peptides containing Pro-Hyp can be obtained, for example, by hydrolysis of collagen or modified collagen such as gelatin with an enzyme, acid, alkali, or the like. The source and production method of collagen peptides are not limited. Artificially synthesized collagen peptides can also be used. One type of collagen peptides may be used alone or two or more types of collagen peptides may be used in combination.

Collagen or gelatin as a raw material of the collagen peptides may be one from bovine, swine, chicken, fish, or the like. One or more of these can be used as raw materials. In one embodiment, collagen from fish is preferred. Fish may be saltwater fish or freshwater fish. Examples include tuna (yellowfin), shark, cod, olive flounder, righteye flounder, sea bream, tilapia, salmon, and catfish.

Any enzyme may be used to prepare the collagen peptides as long as the enzyme can cleave peptide bonds of collagen or gelatin. Examples include collagenase, papain, bromelain, actinidine, ficin, cathepsin, pepsin, chymosin, trypsin, and enzymatic preparations in which these enzymes are mixed. The acid may be, for example, hydrochloric acid, sulfuric acid, nitric acid, or the like. The alkali may be, for example, sodium hydroxide, calcium hydroxide, or the like.

In the present invention, an aqueous solution of hydrolyzed collagen peptides may be used as it is or a hydrolyzed collagen peptide powder obtained by drying or the like may be used. Alternatively, the aqueous solution subjected to a usual purification treatment may be used in the form of an aqueous solution, a powder, or the like.

Commercially available collagen peptides may be used. When the amount of Pro-Hyp in collagen peptides is less than a specific amount, for example, Pro-Hyp may be suitably added.

The average molecular weight of collagen peptides containing Pro-Hyp is not limited. In one embodiment, the average molecular weight of collagen peptides containing Pro-Hyp is preferably 300 or more, more preferably 350 or more, still more preferably 400 or more, yet still more preferably 450 or more, and is also preferably 5000 or less, more preferably 4000 or less, still more preferably 3000 or less, yet still more preferably 2000 or less, particularly preferably 1000 or less. The average molecular weight is preferably 300 to 5000, more preferably 300 to 4000, still more preferably 300 to 3000, yet still more preferably 350 to 2000, particularly preferably 400 to 2000, more particularly preferably 400 to 1000, most preferably 450 to 1000. When the average molecular weight of collagen peptides is in the above ranges, the effect of the present invention can be more sufficiently exhibited.

Herein, the average molecular weight of the collagen peptides is the weight average molecular weight. Herein, the average molecular weight of the collagen peptides means the value measured by a relative molecular mass measurement method in Chinese National Standards (GB standards) GB/T 22729-2008: oligopeptides powder of marine fish. Yet, substitution products are used as reagents for M, 451 and M, 189.

In this method, substances whose molecular weights are known such as cellular pigment C (cytochrome, M, 6500), Trasylol (aprotinin, M, 12500), Bacillus (bacitracin, M, 1450), glycine-glycine-tyrosine-arginine (M, 451), and glycine-glycine-glycine (M, 189) are measured in advance under the same conditions to obtain a relative molecular mass calibration curve showing a relationship between retention time and logarithm of relative molecular weight. The average molecular weight of the collagen peptides is calculated based on the calibration curve. The average molecular weight herein means the weight average molecular weight calculated in terms of each standard substance according to this method.

The amount of Pro-Hyp in the liquid oral composition or collagen peptides can be measured by a method described in Examples, using LC/MS/MS.

In order to suppress foaming, the amount of collagen peptides in the liquid oral composition is preferably 600 mg/100 mL or more, more preferably 1000 mg/100 mL or more, still more preferably 2000 mg/100 mL or more, and is also preferably 110000 mg/100 mL or less, more preferably 20000 mg/100 mL or less, still more preferably 11000 mg/100 mL or less, yet still more preferably 10000 mg/100 mL or less, particularly preferably 7500 mg/100 mL or less. In one embodiment, the amount of collagen peptides in the liquid oral composition is preferably 600 to 110000 mg/100 mL, more preferably 600 to 20000 mg/100 mL, still more preferably 1000 to 11000 mg/100 mL, yet still more preferably 1000 to 10000 mg/100 mL, particularly preferably 2000 to 7500 mg/100 mL. The amount refers to the amount of collagen peptides containing Pro-Hyp, i.e., the total amount of Pro-Hyp and collagen peptides other than Pro-Hyp. The amount of collagen peptides can be measured by a known method, for example, using a device such as LC/MS/MS.

In order to suppress foaming, the amount of Pro-Hyp in collagen peptides in the liquid oral composition is preferably 0.01 wt % or more, more preferably 0.05 wt % or more, still more preferably 0.20 wt % or more, particularly preferably 0.50 wt % or more, and is also preferably 4.0 wt % or less, more preferably 3.5 wt % or less. In one embodiment, in order to suppress foaming, the amount of Pro-Hyp in collagen peptides is preferably 0.01 to 4.0 wt %, more preferably 0.05 to 3.5 wt %, still more preferably 0.20 to 3.5 wt %, particularly preferably 0.50 to 3.5 wt %. When the amount of Pro-Hyp in collagen peptides is in the above ranges, the effect of the present invention can be more sufficiently exhibited.

The liquid oral composition of the present invention may contain one or more components other than collagen peptides as long as the effect of the present invention is not impaired.

The liquid oral composition of the present invention may contain, for examples, one or more additives. Examples thereof include sweeteners (e.g., erythritol, acesulfame K, and sucralose), acidulants (e.g., citric acid), antioxidants, stabilizers, preservatives, flavoring or masking agents, emulsifiers, pigments, seasonings, pH adjusters, nutritional enhancers, and thickening agents (e.g., welan gum and xanthan gum). In addition to Pro-Hyp, the liquid oral composition of the present invention may also contain one or more biofunctional materials such as a material known to have a skin improving effect. Examples of the material known to have a skin improving effect include proteoglycans, elastin peptides, ceramides, plant extracts, chondroitin sulfates, glucosamines, minerals (e.g., calcium), and vitamins (e.g., vitamin C).

The liquid oral composition of the present invention contains an aqueous medium, usually water. Preferably, the liquid oral composition of the present invention is a liquid oral composition (aqueous liquid oral composition) that uses water as a medium.

The pH of the liquid oral composition of the present invention is preferably 6 or less, more preferably 2 to 6, still more preferably 3 to 4.5, for antiseptic properties. The pH herein is the pH at 25° C. The pH can be adjusted using an acid or a salt thereof usable in foods and beverages. One type of acids or salts thereof may be used or two or more types of acids or salts thereof may be used in combination.

Preferably, the liquid oral composition of the present invention is used as a beverage (beverage composition).

The liquid oral composition of the present invention may be a non-carbonated beverage or a carbonated beverage, but is preferably a non-carbonated beverage. A carbonated beverage is a beverage with carbon dioxide gas injected by pressure. A non-carbonated beverage is a beverage without carbon dioxide gas injected under pressure.

The liquid oral composition of the present invention can be packed in a container. In one embodiment, preferably, the liquid oral composition of the present invention is a packaged beverage. The form of the container is not limited. A sealed container such as a bottle, can, plastic bottle, paper pack, aluminum pouch, or plastic pouch can be used to pack the liquid oral composition so as to provide a packaged beverage or the like.

The method of producing the liquid oral composition of the present invention is not limited. For example, the method preferably includes a mixing step of mixing the components. In the mixing step, preferably, the components are mixed by adding an aqueous medium (usually, water). The order of mixing the components is not limited as long as the components are mixed uniformly. The production of the liquid oral composition may include a pH adjusting step of adjusting the pH of the liquid oral composition. The pH adjusting step may be performed simultaneously with or after the mixing step. Steps such as filtration, dilution, and sterilization may be suitably performed, if necessary. When the liquid oral composition is provided as a packaged beverage, a step of filling a container with the liquid oral composition may be performed.

The liquid oral composition of the present invention can be produced by, for example, a method of producing a liquid oral composition, the method including mixing collagen peptides with an aqueous medium so that an amount of Pro-Hyp is 1.0 to 180 mg based on 100 mL of the liquid oral composition. The present invention also encompasses a method of producing the above liquid oral composition. In the above production method, preferably, to 100 mL of an aqueous medium (preferably, water) is added collagen peptides in an amount of 600 to 110000 mg (preferably 600 to 20000 mg, more preferably 1000 to 11000 mg, still more preferably 1000 to 10000 mg, particularly preferably 2000 to 7500 mg). The amount of collagen peptides refers to the amount of collagen peptides containing Pro-Hyp. Collagen peptides, preferred embodiments thereof, and the like are as described above.

The method of adding 1.0 to 180 mg/100 mL of Pro-Hyp to the liquid oral composition is not limited. Examples of the method include one that adds Pro-Hyp, and one that adjusts the amount of Pro-Hyp in collagen peptides for use in preparation of the liquid oral composition. In one embodiment, the amount of Pro-Hyp in the liquid oral composition is preferably 5.0 to 180 mg/100 mL, more preferably 10 to 180 mg/100 mL, still more preferably 10 to 150 mg/100 mL, particularly preferably 40 to 150 mg/100 mL. The production method may include other steps in addition to the mixing step, such as the pH adjusting step and a step of filling a container with the liquid oral composition.

The liquid oral composition of the present invention is rendered less prone to foaming, and for example, it is less likely to bubble when shaken.

Suppression of foaming in the present invention can be evaluated by, for example, vigorously shaking the liquid oral composition and measuring the height of foam (bubble distance) from the liquid level after a certain period of time after shaking.

In the present invention, the amount of Pro-Hyp in the liquid oral composition containing collagen peptides is set to a specific range, whereby foaming, for example, foaming by shaking, can be suppressed. The liquid oral composition of the present invention is rendered less prone to foaming, and is thus advantageous when packed in a container to provide a packaged beverage. The liquid oral composition of the present invention is also less prone to foaming even when shaken during transportation or the like by a vehicle or the like.

### <Method of Suppressing Foaming of Liquid Oral Composition Containing Collagen Peptides>

In another embodiment, the present invention relates to a method of suppressing foaming of a liquid oral composition containing collagen peptides, the method including: adjusting an amount of Pro-Hyp in the liquid oral composition to 1.0 to 180 mg/100 mL.

The amount of Pro-Hyp in the liquid oral composition containing collagen peptides is adjusted to the above range, whereby foaming of the composition can be suppressed.

The method of adjusting the amount of Pro-Hyp in the liquid oral composition is not limited, as long as the liquid oral composition containing collagen peptides ultimately contains the above amount of Pro-Hyp. Examples of the adjustment method include one that adds Pro-Hyp to the liquid oral composition containing collagen peptides, and one that adjusts the amount of Pro-Hyp in collagen peptides for use in preparation of the liquid oral composition.

Collagen peptides, preferred embodiments thereof, the amount of Pro-Hyp, and the like are the same as those in the liquid oral composition of the present invention described above. The liquid oral composition may contain any of the other components described above.

### EXAMPLES

The followings are examples that more specifically describe the present invention. The present invention is not limited to these examples.

In Examples 1 to 8 and Comparative Example 1, collagen peptides containing 0.01 wt % (100 ppm) Pro-Hyp (PO) and having an average molecular weight of 1000 (hereinafter described as “collagen peptides (I)”) were used. The collagen peptides (I) are collagen peptides derived from fish. In Examples 1 to 8, Pro-Hyp (PO) was used in addition to the collagen peptides (I) to prepare liquid oral compositions. Pro-Hyp (PO) available from BACHEM was used. In the examples, PO is linear PO.

The amount of PO in the collagen peptides (I) was analyzed by the following method.

### (Preparation of Sample for Analysis)

A sample was prepared by mixing the collagen peptides (I) (5000 mg) with water (100 mL). The sample was well shaken to obtain a uniform solution, and 100 μL of the solution was dispensed into a 1.5 mL Eppen tube. To the tube was added a 1% aqueous solution of formic acid (900 μL), followed by sufficient mixing in a vortex mixer, whereby a 10-fold dilution of the sample was obtained. Then, 100 μL of the 10-fold dilution was dispensed into another Eppen tube, and a 1% aqueous solution of formic acid (900 μL) was added thereto, followed by sufficient mixing in a vortex mixer, whereby a 100-fold dilution of the sample was obtained. The 100-fold dilution of the sample was filtered through a 0.45 μm membrane filter, and was analyzed under the following LC/MS/MS analysis conditions.

### (Analyzer)

The following device was used.

LC/MS/MS: LCMS-8050 available from Shimadzu Corporation; pump:
LC-30AD available from Shimadzu Corporation; column oven:
CTO-20AC available from Shimadzu Corporation
(LC analysis conditions)
Column: Intrada Amino Acid (Prod No. WAA34, Ser No. PEO9HQF available from Imtakt), 3 μm, 3.0 mm I.D.×100 mm
Column temperature: 35° C.
Flow rate: 0.6 mL/min
Eluent A: acetonitrile with 0.1% formic acid
Eluent B: 100 mM aqueous solution of ammonium formate
Gradient: Eluent B (vol %) 14% (0 min)−14% (6 min)−100% (20 min)−14% (20.1 min)−14% (24 min)
LC end time: 24 min
Amount of injection: 1 μL
Initial pressure after equilibrium: pump A: 6.3 MPa; pump B: 6.4 MPa

### (MS Analysis Conditions)

Ionization mode: ESI Positive
Nebulizer gas flow rate: 3 L/min
Drying gas flow rate: 10 L/min
DL temperature: 250° C.
Block heater temperature: 400° C.
Interface temperature: 300° C.
Heating gas flow rate: 10 L/min
Analysis mode:

### Analysis of PO: MRM (+) 229.10>70.05

### Q1 Pre Bias (V): −30.0; CE: −24.0,

### Q3 Pre Bias (V): −30.0

### Example 1

A liquid oral composition was prepared by mixing PO (0.5 mg) and the collagen peptides (I) (5000 mg) with water (100 mL).

### Examples 2, 3, 4, and 5

Liquid oral compositions were prepared as in Example 1, except that the amount of PO was changed as shown in Table 1. In Example 2, the amount of PO was 39.5 mg. In Example 3, the amount of PO was 79.5 mg. In Example 4, the amount of PO was 99.5 mg. In Example 5, the amount of PO was 179.5 mg.

### Example 6

A liquid oral composition was prepared as in Example 1, except that the amounts of PO and the collagen peptides (I) were changed as shown in Table 1.

### Comparative Example 1

A liquid oral composition was prepared by mixing the collagen peptides (I) (5000 mg) with water (100 mL).

Table 1 shows the amounts of raw materials (PO and the collagen peptides (I)) used to prepare the liquid oral compositions and the amount (concentration) of PO in the liquid oral compositions. The numbers in the parentheses under the amounts of the collagen peptides (I) in Table 1 show the amount of PO (derived from the collagen peptides (I)) in the collagen peptides (I). The amount of PO refers to the amount (mg/100 mL) of PO in the liquid oral composition, and is the total amount of PO used to prepare the composition and PO in the collagen peptides (I). For example, in the case of the liquid oral composition in Example 1, 0.5 mg of PO and 5000 mg of the collagen peptides (I) were used, and the amount of PO in 5000 mg of the collagen peptides (I) was 0.5 mg, so that the total amount of PO in the liquid oral composition was 1.0 mg/100 mL.

The amount of PO in the collagen peptides (the total of PO and the collagen peptides (I)) in each liquid oral composition was as follows: 0.02 wt % in Example 1; 0.79 wt % in Example 2; 1.57 wt % in Example 3; 1.96 wt % in Example 4; 3.48 wt % in Example 5; and 0.18 wt % in Example 6.

Regarding the foaming of the liquid oral compositions obtained in the examples and the comparative examples, the following method was used to evaluate how easily the liquid oral composition bubbles when shaken. Table 1 shows the results.

### <Evaluation of Foaming>

The liquid oral composition (50 mL) was placed in a 100 mL stoppered calorimetric tube (available from Sansho Co., Ltd., Tokyo, with scale (cylindrical shape with an internal diameter of 2.5 cm)), and the tube was plugged. The scale of the glass bottle (stoppered colorimetric tube) was graduated in increments of 1 mL.

The glass bottle was vigorously shaken up and down for 20 times, and the height of foam from the liquid level (bubble distance) (on the scale) three minutes after shaking was visually checked. The height of the liquid level and the bubble distance were read on the scale of the glass bottle (the bottom corresponds to zero on the scale). Table 1 shows the numbers on the scale as evaluation results.

TABLE 1

| | | | | | | | | Comparative |
|---|---|---|---|---|---|---|---|---|
| | | Example 1 | Example 2 | Example 3 | Example 4 | Example 5 | Example 6 | Example 1 |
| Amounts of | Collagen peptides (I) | 5000 | 5000 | 5000 | 5000 | 5000 | 100000 | 5000 |
| raw materials | (amount of PO in | (0.5) | (0.6) | (0.5) | (0.6) | (0.5) | (10.0) | (0.5) |
| (mg/100 mL) | parentheses) | | | | | | | |
| | PO | 0.6 | 39.5 | 79.6 | 99.5 | 179.5 | 170.0 | 0 |
| Amount of PO (mg/100 mL) | | 1.0 | 40.0 | 80.0 | 100.0 | 180.0 | 180.0 | 0.6 |
| Evaluation | Height of liquid level | 50 | 60 | 50 | 60 | 50 | 50 | 50 |
| results | Bubble distance | 44 | 32 | 28 | 17 | 13 | 40 | 48 |

Table 1 shows the height of the liquid level in terms of the distance (height) from the bottom of the glass bottle to the liquid level (number on the scale) three minutes after shaking. The bubble distance is the height of bubbles from the liquid level (number on the scale) three minutes after shaking. For example, in Example 1, the distance (height) from the bottom of the glass bottle to the liquid level was 50 on the scale and the height of bubbles from the liquid level was 44 on the scale three minutes after shaking. In other words, in Example 1, the highest height of bubbles from the bottom of the glass bottle three minutes after shaking was 94 on the scale.

### Examples 7 and 8

Liquid oral compositions were prepared as in Example 1, except that the amounts of Pro-Hyp (PO) and the collagen peptides (I) were changed as shown in Table 2. In Example 7, the amount of PO was 9.5 mg. In Example 8, the amount of PO was 149.5 mg. The amount of PO in the collagen peptides (the total of PO and the collagen peptides (I)) in the liquid oral composition was 0.20 wt % in Example 7 and 2.91 wt % in Example 8.

In Examples 9 and 10, liquid oral compositions were prepared using the later-described collagen peptides (II) or (III) and the above-described PO (available from BACHEM). The amount PO in the collagen peptides (II) or (III) was determined by the same method used for the collagen peptides (I).

### Example 9

Collagen peptides containing 1 wt % Pro-Hyp (PO) and having an average molecular weight of about 500 (described as “collagen peptides (II)”) was used. The collagen peptides (II) are collagen peptides derived from fish. A liquid oral composition was prepared by mixing PO (available from BACHEM) (30.0 mg) and the collagen peptides (II) (5000 mg) with water (100 mL). The amount of PO in the collagen peptides (the total of PO and the collagen peptides (II)) in the liquid oral composition was 1.59 wt %.

### Example 10

Collagen peptides containing 0.0015 wt % Pro-Hyp (PO) and having an average molecular weight of about 2000 (described as “collagen peptides (III)”) was used. The collagen peptides (III) are collagen peptides derived from swine. A liquid oral composition was prepared by mixing PO (available from BACHEM) (49.93 mg) and the collagen peptides (III) (5000 mg) with water (100 mL). The amount of PO in the collagen peptides (the total of PO and the collagen peptides (III)) in the liquid oral composition was 0.99 wt %.

Regarding the foaming of each of the liquid oral compositions obtained in Examples 7 to 10, the above-described method was used to evaluate how easily the liquid oral composition bubbles when shaken. Table 2 shows the results.

The numbers in the parentheses under the amounts of the collagen peptides (I) to (III) in Table 2 show the amounts of PO (derived from the collagen peptides (I) to (III)) in the collagen peptides (I) to (III). The amount of PO shown in Table 2 refers to the amount of PO (mg/100 mL) in the liquid oral composition, and is the total amount of PO used to prepare the composition and PO in the collagen peptides (I) to (III). For example, in the case of the liquid oral composition in Example 9, 30.0 mg of PO and 5000 mg of the collagen peptides (II) were used, and the amount of PO in 5000 mg of the collagen peptides (II) was 50.0 mg, so that the total amount of PO in the liquid oral composition was 80.0 mg/100 mL.

TABLE 2

| | | Example 7 | Example 8 | Example 9 | Example 10 |
|---|---|---|---|---|---|
| Amounts of | Collagen peptides (I) | 5000 | 5000 | — | — |
| raw | | (0.5) | (0.5) | | |
| materials | Collagen peptides (II) | — | — | 5000 | — |
| (mg/100 mL) | | | | (50.0) | |
| | Collagen peptides (III) | — | — | — | 5000 |
| | | | | | (0.07) |
| | PO | 9.5 | 149.5 | 30.0 | 49.93 |
| Amount of PO (mg/100 mL) | | 10.0 | 150.0 | 80.0 | 50.0 |
| Evaluation | Height of liquid level | 50 | 50 | 50 | 50 |
| results | Bubble distance | 40 | 16 | 5 | 38 |

As in Table 1, Table 2 shows the height of the liquid level in terms of the distance (height) from the bottom of the glass bottle to the liquid level (number on the scale) three minutes after shaking. The bubble distance is the height of bubbles from the liquid level (number on the scale) three minutes after shaking.

When the amount of PO was 1.0 to 180.0 mg/100 mL, the foaming of liquid oral composition was suppressed.

### INDUSTRIAL APPLICABILITY

The present invention is useful in the food and beverage field and the like.

1. A liquid oral composition comprising:
collagen peptides containing Pro-Hyp,
wherein an amount of Pro-Hyp is 1.0 to 180 mg/100 mL.

2. The liquid oral composition according to claim 1,
wherein the amount of Pro-Hyp is 10 to 180 mg/100 mL.

3. The liquid oral composition according to claim 1,
wherein the liquid oral composition is a packaged beverage.

4. A method of suppressing foaming of a liquid oral composition containing collagen peptides, the method comprising:
adjusting an amount of Pro-Hyp in the liquid oral composition to 1.0 to 180 mg/100 mL.

5. A method of producing a liquid oral composition, the method comprising:
mixing collagen peptides with an aqueous medium so that an amount of Pro-Hyp is 1.0 to 180 mg based on 100 mL of the liquid oral composition.
